(19) United States
(12) Patent Application Publication
(10) Pub. No.: US 2006/0240485 A1
(43) Pub. Date: Oct. 26, 2006

(54) Method of monitoring immunotherapy

(75) Inventors: Christoph Hock, Erlenbach (CH); Uwe Konietzko, Zürich (CH); Roger Nitsch, Zumikon (CH)

Correspondence Address: STERNE, KESSLER, GOLDSTEIN & FOX PLLC, 1100 NEW YORK AVENUE, N.W., WASHINGTON, DC, 20005, US

(73) Assignee: Universitat Zurich, Zurich (CH)

(21) Appl. No.: 10/554,314

(22) PCT Filed: Oct. 15, 2003
(86) PCT No.: PCT/EP03/11413

§ 371 Date: Apr. 19, 2006

Related U.S. Application Data
(60) Provisional application No. 60/464,888, filed on Apr. 24, 2003.

(51) Int. Cl. G01N 33/567; G01N 33/53

(52) U.S. Cl. 435/7.2

(57) ABSTRACT

The present invention relates to a method of monitoring an immunotherapy against amyloidosis and other diseases characterized by the deposition of abnormal protein aggregates. More specifically, it relates to a method of evaluating an immunotherapy against Alzheimer's disease, based on a novel assay that is characterized by scoring immunoreactivity levels of patient sera in amyloid plaque containing samples. The assay possesses highly predictive properties in relation to the clinical outcome of such an immunotherapy, in contrast to previously used, conventional ELISA assays. Therefore, the novel assay is useful for the evaluation of the efficacy of an immunotherapy in a patient suffering from amyloidosis, particularly Alzheimer's disease.

The present invention relates to a method of monitoring an immunotherapy against amyloidosis and other diseases characterized by the deposition of abnormal protein aggregates. More specifically, it relates to a method of evaluating an immunotherapy against Alzheimer's disease, based on an assay for scoring immunoreactivity levels of patient sera in amyloid plaque containing samples.

Beta-amyloid is a major histopathological hallmark of Alzheimer's disease (AD). It is associated with age-related cognitive decline (Naslund et al., 2000; Chen et al., 2000), age-related neurotoxicity (Geula et al., 1998), and with the formation of neurofibrillary tangles (Götz et al., 2001; Lewis et al., 2001). Therefore, several β-amyloid-lowering strategies are currently developed for clinical use. These include inhibition of the generation of amyloid β-peptide (Aβ) with β- and γ-secretase inhibitors, prevention of Aβ aggregation, and immunization against β-amyloid (Citron, 2002; Weiner and Selkoe, 2002; Sigurdsson et al., 2002; Gandy, 2002). Both passive and active immunization of transgenic mice against β-amyloid can reverse neuropathology and improve pathologic learning and memory behaviors (Schenk et al., 1999; Bard et al., 2000; Janus et al., 2000; Morgan et al., 2000; De Mattos et al., 2001). It is still unknown whether antibodies against μ-amyloid can also modify pathology in human patients with AD. A recent neuropathologic examination of one patient with AD who received Aβ immunization revealed highly unusual histology: Despite the fact that the histopathological criteria for AD were met for this case, large brain areas were devoid of β-amyloid, and were associated with reduced neuritic pathology and with reduced astrocytosis. Notably, in the brain areas with low β-amyloid load, microglial cells were found to be filled with fl-amyloid, a highly unusual finding (Nicoll et al., 2003).

Whether active immunization can slow the progression of dementia in patients with AD was recently tested in a multicenter Phase 2A study. Active dosing of the vaccine, however, was suspended after the occurrence of clinical signs of post-vaccination aseptic menigoencephalitis in 6% of the immunized cases (Schenk et al., 2002). A detailed account of these cases was report by Orgogozo et al., 2003.

Currently, there is no method available to measure and to monitor the outcome of an immunotherapy as described above. It was tested whether the generation of antibodies against β-amyloid is effective in slowing progression of Alzheimer's disease, and we assessed cognitive functions in 30 patients who received a prime and a booster immunization of aggregated Aβ₄₂ over a one year period in the Zurich cohort of a placebo-controlled randomized multicenter trial. Twenty of 30 patients generated antibodies against β-amyloid as determined by the tissue amyloid plaque immunoreactivity (TAPIR) assay of the present invention. Patients who generated such antibodies showed significantly slower rates of decline of both cognitive functions and activities of daily living as indicated by the Mini Mental State Examination, the Disability Assessment for Dementia and the visual paired delayed recall test from the Wechsler Memory Scale, as compared to patients without such antibodies. These beneficial clinical effects associated with the generation of antibodies against β-amyloid were also present in two of three patients who had experienced transient episodes of immunization-related aseptic meningoencephalitis. These findings establish that the generation of antibodies against β-amyloid plaques can slow cognitive decline in patients with Alzheimer's disease. Importantly, the analyses of antibody titers measured by ELISA failed to predict the clinical outcome. Therefore, it is an object of the present invention to provide a novel tissue amyloid plaque immunoreactivity (TAPIR) assay and the use thereof. This assay is suited inter alia for the analysis of multicenter cohorts of immunizations trials, and it is especially useful to monitor and evaluate the efficacy of an immunotherapy in patients suffering from a neurodegenerative disease associated with the deposition of abnormal protein aggregates and/or amyloidosis, in particular Alzheimer's disease. This object has been solved by the features of the independent claims. The subclaims define preferred embodiments of the present invention.

By using a specific and sensitive tissue amyloid plaque immunoreactivity (TAPIR) assay, according to the present invention, it was possible to observe the sustained generation of antibodies against β-amyloid on brain sections from transgenic mice in 20 of 30 of these patients. To determine whether these antibodies were associated with modifications of the clinical course of AD, we tested cognitive functions and capacities of daily living of the patients at baseline (n=30) and during a one year period (n=28, 2 drop outs). Patients with a clinical diagnosis of mild to moderate AD had received active prime and booster immunizations with pre-aggregated Aβ₄₂(QS-21) (n=24) or placebo (n=6) in a double-blind, randomized study design (Hock et al., 2002; Schenk et al., 2002; Orgogozo et al., 2003).

The singular forms “a”, “an”, and “the” as used herein and in the claims include plural reference unless the context dictates otherwise. For example, “a sample” means as well a plurality of samples, and so forth. The term “and/or” as used in the present specification and in the claims implies that the phrases before and after this term are to be considered either as alternatives or in combination. Neurodegenerative diseases or disorders associated with the deposition of abnormal protein aggregates according to the present invention comprise amyloidogenic diseases, in particular Alzheimer's disease, whereby the term ‘AD’ shall mean Alzheimer's disease, Parkinson's disease, Huntington's disease, amyotrophic, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Further conditions involving the deposition of abnormal protein aggregates are, for instance, age-related macular degeneration and prion diseases.

In one aspect, the invention provides a method of monitoring an immunotherapy, of measuring and of prognosticating the outcome of an immunotherapy in a subject which may suffer from a neurodegenerative disease which is associated with the deposition of abnormal protein aggregates. The method comprises: (a) obtaining a sample from a subject being immunized against a component of said abnormal protein aggregate, said sample will be the test sample, (b) contacting said test sample with a sample containing an abnormal protein aggregate, (c) determining the level of immunoreactivity of said test sample against abnormal protein aggregates in said abnormal protein aggregate-containing sample, and (d) comparing said level of immunoreactivity to a reference value, whereby said reference value represents a known disease or health status, or the status prior to onset of said immunotherapy in said subject. An increase in the level of immunoreactivity of said test sample from said subject undergoing immunotherapy is indicative of a positive clinical outcome of said immunotherapy.

In a preferred embodiment of the herein claimed method of monitoring an immunotherapy, of measuring and of prognosticating the outcome of an immunotherapy said abnormal protein aggregate-containing sample is obtained from a transgenic non-human animal and in a further preferred embodiment said abnormal protein aggregate-containing sample is a tissue. section from a non-human animal. Said non-human animal being transgenic for a human protein, or a fragment, or derivative, or a mutant thereof, wherein said human protein is a component of said abnormal protein aggregate. The expression of said transgene results in said non-human animal exhibiting a predisposition to developing abnormal protein aggregates.

In another aspect, the invention provides a method of monitoring anrt immunotherapy, of measuring and of prognosticating the outcome of an immunotherapy in a subject which may suffer from an amyloidogenic disease. The method comprises: (a) obtaining a sample from a subject being immunized against an amyloid component, said sample will be the test sample, (b) contacting said test sample with a sample containing amyloid aggregates and/or amyloid plaques, (c) determining the level of immunoreactivity of said test sample against amyloid aggregates and/or against amyloid plaques in said amyloid aggregates and/or amyloid plaques containing sample, and (d) comparing said level of immunoreactivity to a reference value, whereby said reference value represents a known disease or health status, or the status prior to onset of said immunotherapy in said subject. An increase in the level of immunoreactivity of said test sample from said subject undergoing immunotherapy is indicative of a positive clinical outcome of said immunotherapy. The wordings amyloidogenic aggregates, amyloidogenic plaques may be used instead of amyloid aggregates, amyloid plaques but may be tantamount.

In a preferred embodiment of the herein claimed method of monitoring an immunotherapy, of measuring and of prognosticating the outcome of an immunotherapy said amyloid plaque-containing sample is obtained from a transgenic non-human animal and in a further preferred embodiment said amyloid plaque-containing sample is a tissue section from a transgenic non-human animal. In still a further preferred embodiment said amyloid plaque-containing sample is a brain tissue section from a non-human animal. Said non-human animal being transgenic for human amyloid precursor protein (APP), or a fragment, or derivative, or a mutant thereof, and the expression of said transgene results in said non-human animal exhibiting a predisposition to developing amyloid plaques.

In a further aspect of the herein claimed method said amyloid component, also named amyloidogenic component is β-amyloid.

In a further preferred embodiment of the herein claimed methods, kits, assays and uses of the instant invention, said amyloidogenic disease or disorder is Alzheimer's disease and said subject which may suffer from an amyloidogenic disease or disorder may suffer from Alzheimer's disease.

It is particularly preferred that said sample from a subject being immunized against an amyloid component or being immunized against a component of an abnormal protein aggregate, it is said the test sample, is selected from the group comprising a body fluid, which may be cerebrospinal fluid or serum or other body fluids including saliva, urine, blood or mucus. Preferably, the method of monitoring an immunotherapy, of measuring and of prognosticating the outcome of an immunotherapy according to the instant invention, can be practiced ex corpore, and such methods preferably relate to samples, for instance, body fluids or cells or tissues removed, collected, or isolated from a subject or patient or animal.

The novel tissue amyloid immunoreactivity assay, as disclosed in the present invention, shall be referred to as TAPIR assay. Said TAPIR assay was applied to the Zurich cohort of 30 patients who participated in a multicenter trial of β-amyloid immunization. By using said TAPIR assay, a slowed cognitive decline in AD patients who generated antibodies against β-amyloid plaques, could be observed, whereas cognitive measures in patients who did not generate antibodies against β-amyloid worsened. Patients with intermediate increases in antibodies against β-amyloid declined only marginally, and patients with strong increases remained stable. This cognitive stabilization was further substantiated by significantly better performance in activities of daily living and by tests of hippocampal memory functions. These data establish the possibility that antibodies against β-amyloid are clinically effective in halting the progression of AD.

In still another aspect, the invention features a kit for monitoring an immunotherapy, for measuring and for prognosticating the outcome of an immunotherapy, in a subject suffering from a neurodegenerative disease associated with the deposition of abnormal protein aggregates, said kit comprising:
at least a solid phase that contains on its surface an abnormal protein aggregate-containing sample.

It is preferred that the abnormal protein aggregate-containing sample in said kit is obtained from a transgenic non-human animal and it is further preferred that said abnormal protein aggregate-containing sample is a tissue section from a transgenic non-human animal.

In a further preferred embodiment said abnormal protein aggregate-containing sample is a tissue section from a non-human animal transgenic for a human protein, or a fragment, or derivative, or mutant thereof, wherein said human protein is a component of said abnormal protein aggregate, and wherein the expression of said transgene results in said non-human animal exhibiting a predisposition to developing abnormal protein aggregates.
It may be further preferred that said human protein is the amyloid precursor protein, APP, or a fragment, or derivative, or mutant thereof.
In a further aspect, the kit is for monitoring an immunotherapy, for measuring and for prognosticating the outcome of an immunotherapy, in a subject suffering from a neurodegenerative disease wherein said neurodegenerative disease is preferably an amyloidogenic disease or disorder, and wherein said amyloidogenic disease is preferably AD.
In still a further aspect, said abnormal protein aggregate-containing sample of said kit comprises amyloid plaques or amyloidogenic components and in a further preferred embodiment said amyloid plaques or amyloidogenic components contain β-amyloid.

Notably, the TAPIR scores of the immune sera as determined by analyzing human β-amyloid on brain sections of transgenic mice were more predictive for the therapeutic outcome than antibody titers measured by ELISA. This may be related to clinically important qualitative characteristics of the antibodies with respect to epitope recognition, affinity and avidity of the antibodies to react with bona fide human β-amyloid generated slowly over time in the physiologic brain environment—as opposed to artificial binding conditions of the antibodies to Aβ immobilized on plastic ELISA plates. Despite the fact that the TAPIR scores were statistically correlated with ELISA titers of serum antibodies against Aβ₄₂ (rₛ=0.700, p<0.001), there was a subgroup of patients with widely discrepant results of these two measures, suggesting that the degree of selectivity of the antibodies for bona fide human β-amyloid. is an important determinant for the clinical efficacy of immunotherapy of AD.

The observed clinical differences among AD patients with and without the generation of antibodies against β-amyloid were unrelated to the AChEI treatments, because 28 of 30 patients included in this study were on stable dosages of AChEI before and during this trial. These data therefore support the possibility that the therapeutic effects of antibodies against β-amyloid and AChEI are additive. For the formal test of this possibility, however, control groups without AChEI treatments are required. Other factors that could potentially affect rates of progression of dementia including age, gender, medication and head trauma were either excluded by the selection criteria or were distributed evenly among the groups with and without antibodies against β-amyloid. The ApoE genotype affects the risk for getting AD as well as the age of onset, but not the rate of cognitive decline one the disease has started (Growdon et al., 1996). Nevertheless, the distribution of the common ApoE genotypes was equal among our groups (p=0.114, χ²=2.5; d.f.=1 for genotypes, and p=0.438, χ²=0.602; d.f.=1 for allele frequencies), and there was no carrier of an ApoEε2 allele in our study cohort.

During the course of the AN1792 multicenter trial, 6% of the study patients developed clinical signs of aseptic meningoencephalitis (Schenk, 2002), and were generally treated with corticosteroids. These signs were not correlated with the generation of antibodies against β-amyloid. Moreover, occurrence of aseptic meningoencephalitis did not predict clinical outcome: Two patients with aseptic meningoencephalitis and who generated antibodies against β-amyloid in our cohort remained cognitively stable one year after the immunizations, despite the transient and reversible drop during the acute symptoms. On the other hand, dementia severity in one other patient with aseptic meningoencephalitis and without antibodies against β-amyloid continuously declined after recovery from the acute symptoms. These data imply the possibility that the beneficial effects of antibodies against β-amyloid on cognitive measures are maintained even after transient episodes of post-vaccination aseptic meningoencephalitis.

Previous passive immunization studies of transgenic mice with a monoclonal antibody against soluble Aβ resulted in increased plasma and CSF levels of Aβ within 24 to 72 hours (Dodart et al., 2002; De Mattos et al., 2002). These data gave rise to the interpretation that binding of an antibody to plasma Aβ can lead to sequestration of Aβ followed by a net efflux of soluble Aβ from brain to plasma. On the other hand, high affinity binding of antibodies to Fc receptors is important for their ability to remove brain β-amyloid in mice, suggesting an important role of Fc receptor-mediated uptake of β-amyoid by macrophages or microglial cells (Bard et al., 2003). The unchanged plasma levels of Aβ in our human study argues against sequestration of plasma Aβ as an underlying principle of the observed therapeutic effects. Importantly, the antibodies against β-amyloid reported here are substantially different to the monoclonal antibodies used in the mouse studies, because the human immune sera failed to react with soluble Aβ but readily reacted with structural epitopes of β-amyloid in plaques and in vascular structures (Hock et al., 2002).

The results of the study undelying the present invention may affect the status of the amyloid cascade hypothesis of AD. Current versions of the amyloid cascade hypothesis claim a primary role of β-amyloid in the pathogenesis of AD (for reviews, see Steiner and Haass, 2000; Selkoe, 2001; Walter et al., 2001; Hardy and Selkoe 2002; Selkoe, 2002; Golde, 2002; Ingelson and Hyman 2002; Dominguez and De Strooper, 2002; Sisodia and St. George-Hyslop, 2002). In analogy to infectious disease, where the primary role in causing disease is played by an infectious agent, the characterization of the pathogenic mechanism of AD can be accomplished by two powerful and complementary experimental approaches: Transmission and vaccination. Transmission experiments are designed to identify the disease-causing entity—e.g. a virus—in a diseased tissue by isolating the minimal disease-causing entity from irrelevant contaminants, by transmitting it to a healthy animal, and by thereby causing the disease phenotype. To a large extent, this was accomplished for β-amyloid by two independent experiments in transgenic mice. These experiments have shown neurofibrillar degeneration along with the formation of bona fide neurofibrillary tangles (NFT) as a result of either intracerebral microinjections of β-amyloid into P301L tau transgenic mice or by transgenic generation of β-amyloid in a P301L mutant background (Götz et al., 2001; Lewis et al., 2001). This was the first time that a role of β-amyloid in the generation of NFT in an animal was recapitulated experimentally.

Vaccination provides a complementary immunological experimental approach to prove a central role of a suspected disease-causing entity. The experiment uses parts of the suspected disease-causing entity as a vaccine to stimulate the immune system of a host animai to produce antibody-mediated immunity. If the antibodies generated against the suspected disease-causing entity can protect against disease—after exposure to an otherwise pathogenic dose of the disease-causing entity—the central role of the disease-causing entity in the disease mechanism is confirmed. From this point of view, the use of β-amyloid as a vaccine tests the possibility that β-amyloid plays a central role in causing cognitive decline in AD. The results, as reported in the present invention, that precisely the patients who developed antibodies against β-amyloid—but not patients without antibodies or with Aβ antibodies that failed to recognize β-amyloid—prevented the progression of AD is therefore the first successful clinical evidence of a central role of β-amyloid in causing cognitive decline and dementia in AD patients. The fact that the degree of the protective effects was related to the degree by which the antibodies reacted with β-amyloid in brain tissue underscores this conclusion.

Important open questions include the relationship of the clinical efficacy to the histopathology following β-amyloid immunization. A recent single case report of highly unusual pathology observed in an immunized patient with AD suggested removal of β-amyloid by microglial cells in large areas of the brain—with normal amounts of NFT throughout the brain (Nicoll et al., 2003). This observation is clearly supportive of the idea that antibody-mediated removal of β-amyloid occurred in response to β-amyloid immunization, but additional histopathological analyses are required to conclusively confirm that removal of β-amyloid from brain is both necessary and sufficient for clinical efficacy.

The results as described in the present invention establish the possibility that antibodies against β-amyloid plaques can slow cognitive decline in patients with AD. For the prediction of clinical outcome, our data establish the use of a TAPIR assay, because of its advantages over conventional ELISA titer assays. Our findings strongly suggest to extend these subset analyses by long-term follow-up studies of the complete cohort of immunized patients who generated antibodies against β-amyloid.

Other features and advantages of the instant invention will be apparent from the following description of examples and figures which are illustrative only and not intended to limit the remainder of the disclosure in any way.

### EXAMPLE 1

Methods

Patients and treatments: The experiments reported here were done within an additional adjunct study of the Zurich cohort of 30 AD patients who participated in the ELAN/Wyeth-Ayerst AN1792(QS-21) multicenter trial. This study was approved by the ethics committee and written informed consent was obtained from all patients and caregivers. The clinical diagnosis of probable AD was made according to the NINCDS-ADRDA criteria (McKhann et al., 1984), and clinically relevant other diseases were excluded. A baseline MRI was done to support the diagnosis of AD and to exclude other structural causes of dementia. Patients with mild to moderate dementia severity assessed by MMSE (Folstein et al., 1975) scores ranging from 15 to 26 points were eligible to participate in this study. The 30 patients in the Zurich cohort had scores of 21.0±3.2 points (S.D.; range=16-26 points) and disease durations of 3.6±2.3 years (mean±S.D.; range=1-11 years). Additional criteria for inclusion included Rosen Modified Ischemic scores of smaller than 5 points to exclude vascular dementia. There were 9 female and 21 male-patients in the Zurich cohort. Their mean age was 72.1±7.2 years (S.D.; range=57 to 81 years). The patients were randomized in a double-blind study design; 24 patients received the active vaccine consisting of pre-aggregated synthetic Aβ₄₂ along with the surface-active saponin QS-21 as an adjuvant, and 6 patients received placebo. Both the active vaccine and placebo were given as a prime intramuscular injection followed one month later by a boost intramuscular injection. The drug/placebo status remained blinded to patients, caregivers, clinical raters and laboratory investigators. One patient from the placebo group died during the study from cerebrovascular hemorrhage. One patient refused to participate in the neuropsychological tests at month 12. Therefore, the study underlying the present invention started with 30 patients at baseline, and ended with 28 patients after the one year observation period. Out of 30 study patients, 28 received stable dosages of AChEI for at least 3 months prior to immunization, and these treatments were continued throughout the study, except for one patient who generated antibodies against β-amyloid and who terminated the AChEI treatment at month 11. In particular, in the group of patients who generated antibodies against β-amyloid, 6 patients received donepezil (5 mg per day, n=1 and 10 mg per day, n=5), 2 patients received rivastigmine (12 mg per day, n=1, 3 mg per day, n=1), 11 patients received galantamine (16 mg per day, n=5, 24 mg per day, n=6), and one patient changed from galantamine (16 mg per day) to rivastigmine (6 mg per day). In the group of patients without antibodies, 5 patients received donepezil (10 mg per day, n=5), 4 patients received galantamine (16 mg per day, n=1, 24 mg per day, n=3), and one patient in this group received no AChEI. The length of treatment with AChEI prior to neuropsychological testing at month 12 was not significantly different across the patient groups with strong increases in TAPIR scores (3.0±2.2 years; mean±S.D.), with intermediate increases (2.1±0.8 years) and without increases (3.6±1.9 years) (p=0.211, Kruskal-Wallis test). These time periods were fairly beyond the one year period of known cognitive stabilizing effects of AChEI (Giacobini et al., 2000). Other non-prescription or prescription medications other than acetylcholinesterase inhibitors for cognitive enhancement were neither permitted within the trial nor within the three-month period prior to inclusion. The use of non-steroidal anti-inflammatory drugs (NSAID), statins, estrogens or vitamin E was permitted both as single medication and in combinations. The use of these drugs was evenly distributed among the two groups with and without antibodies against β-amyloid. In particular, patients who generated antibodies against β-amyloid used NSAIDs (n=11), statins (n=3) vitamin E (n=2), and no estrogens; patients who did not generate antibodies against β-amyloid used NSAIDs (n=5), statins (n=2), vitamin E (n=1) and estrogens (n=2). The average rate of decline of −6.3±4.0 per year (mean±SD) points on the MMSE scale in our group of patients without antibodies against β-amyloid was more pronounced than the 3 to 4 points generally reported for the natural history of large populations of AD patients. This difference could by due to the small number (n=9) of patients in our group without antibodies against β-amyloid. Nevertheless, the average rate of decline of −1.4±3.5 per year in n=19 patients with antibodies against β-amyloid is significantly lower that observed in studies of large populations of patients with AD.

Tissue amyloid plaque immunoreactivity (TAPIR) assay: For the assessment of the ability of the human immune sera to react with bona fide β-amyloid plaques in brain tissue, a specific TAPIR assay, as disclosed in the present invention, was developed. Double transgenic mice (18 months old) expressing human APP and PS1 genes with pathogenic AD-causing mutations (APP^{SW}xPS1^{M146L}) were perfused and brains were fixed. Paraffin-embedded brains were sectioned (5 μm) and incubated with human serum or CSF samples taken prior to the prime injection and 56.0±5.8 days (mean±S.D.) after the booster injection. Samples were used either undiluted or diluted 1:50 to 1:10,000 in 2% BSA and 5% donkey serum in PBS. After washing, human IgG bound to β-amyloid plaques were detected with cy3-conjugated donkey antibodies directed against heavy and light chains of human IgG (Jackson Labs, Bar Harbor, Me.). Fluorescent β-amyloid plaques on the sections were imaged through a 40× objective and a TRITC filter attached to a Nikon Eclipse E800 fluorescence microscope equipped with a Kappa PS 30C CCD camera. Images of all dilutions were acquired with standardized camera settings chosen to be well below the saturation of 255 arbitrary units (A.U.) in 8 bit mode. The Image J software (www.ncbi.nlm.nih.gov) was used to quantify the mean pixel intensities (range: 23 to 195 A.U.) of n=15 β-amyloid plaques per serum dilution. Averages of the means were used for both the standard curve and the individual samples. The assay was linear for serum dilutions ranging from 1:50 to 1:10,000 (r=0.951; p<0.013). For comparisons with a standard curve obtained by diluting human CSF from a responder, both pre-immune and immune serum samples were used at 1:50 dilutions and categorized by two independent and blind raters into the following 5 immunoreactivity scores: absent immunoreactivity (−); weak immunoreactivity corresponding to 1:10,000 (+), moderate, 1:5,000, (++); strong, 1.1,000, (+++); very strong, 1:500 (++++). To determine the increase in immunoreactivity during treatment, the pre-immune immunoreactivity scores were subtracted from the immune scores to generate the following.groups: no increase: n=10 including one death in placebo group equals n=9 observed cases in non-responder group. In the responder group (n=20), one patient dropped out because he was unwilling to participate in neuropsychological testing at month 12, leaving n=19 observed cases. To compare the degree of the immune response to the clinical outcome, this group was further subdivided into two groups bases upon the degree of increases in immunoreactivity scores as follows: Strong increases representing 4+ increases from pre-immune to immune status (n=6), and moderate increases representing the remaining group of 1+ to 3+ increases (n=13) from pre-immune to immune status.

Neuropsychology: Clinical assessments included neuropsychological tests that were obtained at baseline (month 0) as well as months 6 and 12. The cognitive test batteries comprised the Mini Mental State (MMSE), the Alzheimer's Disease Assessment Scale (ADAS) cognitive part (ADAS-Cog) (Rosen et al., 1984), tests from the Wechsler Memory Scale (verbal and visual paired associated immediate and delayed recall) (Wechsler et al., 1987) naming and fluency (verbal and categorial) (CERAD) (Morris et al., 1998). Global function was determined by the clinical dementia rating scale (CDRS) (Morris 1993), as well as the clinical global impression of change (CGIC) (Knopman et al., 1994). Activities of daily living were assessed by Disability Assessment for Dementia (DAD) (Gauthier et al., 2001) rating scale. Normal MMSE scores were assumed at 27 to 30; mild dementia corresponded to 20 to 26; moderate to 14 to 19; and severe dementia to 0 to 13. The DAD rating ranges from 0 to 40 (maximum). The range of the visual paired associated delayed recall test from the Wechsler Memory Scale is 0 to 6. Because of the inherent difficulties of this task, 12 patients were unable to complete this test at 12 months after the start of this trial, respectively. The test scores at baseline for the patients who dropped out of this test were 1.6±1.2 points (n=12), as compared to 2.8±1.5 (n=18). The clinical raters remained blinded throughout the study to the treatment status, as well as to the immunoreactivity scores and antibody titers of the patients.

Titer assays: Antibody titers were measured by ELISA. In brief, blocked Aβ₄₂-coated (Bachem, Weil am Rhein, Germany) microplates (Nunc Maxisorp, Roskilde, Denmark) were incubated with diluted serum samples overnight at 4° C., washed and incubated individually with goat anti-human biotinylated IgG or IgM (H+L) (Jackson Labs, Bal Harbor, Me.), detected by peroxidase-conjugated streptavidin (Jackson Labs, Bal Harbor, Me.) and 3,5,3′,5′-tetramethylbenzidine (TMB) (Sigma) at 450 nm on a microplate reader (Victor2 Multilabel, EG&G® Wallac). All samples and standards were assayed in duplicates.

Aβ₄₂ and Aβ₄₀ ELISAs: CSF and plasma Aβ₄₂ were measured by ELISA (INNOTEST β-Amyloid 1-42, Innogenetics, Belgium) according to the manufacturer's protocol. CSF Aβ₄₀ ELISA: 1 82 g/ml of biotinylated 4G8 (Signet, Dedham, Mass.) was bound to streptavidin-coated microplates (Nunc) and incubated with CSF diluted in PBS, along with BAP-24 (courtesy of Dr. Manfred Brockhaus, Roche), followed by TMB as the chromophor, sulfuric acid and reading at 450 nm. Standard curves of Aβ₄₀ (Bachem) scaling from 0.15 to 40 ng/ml were used, and Aβ₄₂ was tested as a negative control.

Statistical analyses: Data were analyzed by analysis of variance (ANOVA). Comparisons of two groups were done with Mann-Whitney U tests, and comparisons of three groups were done by Kruskal-Wallis tests. The distribution of categorial variables between groups was tested by using the chi-square and Fisher's exact tests. The correlation coefficient quoted is Spearman's rho. All p values reported are two-sided. Changes in neuropsychological test scores (three data collection time points) were analysed by observed cases analysis (OC). Changes in serum titers and plasma Aβ levels (ten data collection time points) were analysed by intention to treat (ITT) analysis; missing values were interpolated between visits and last values were carried forward.

### RESULTS

Human antibodies specifically recognized brain β-amyloid plaques: Twenty of 30 patients in the study reported herein generated antibodies that specifically recognized β-amyloid plaques on brain tissue sections obtained from transgenic mice expressing in brains both human APP with the Swedish mutation and human presenilin. 1 (PS1) with the M146L mutation (APP^{Sw}xPS1^{M146L}) (Holcomb et al., 1998) (FIG. 1). The presence or the absence of these antibodies against β-amyloid was unrelated to the occurrence of aseptic meningoencephalitis in 3 of 30 immunized patients. Confocal microscopy images of β-amyloid plaques stained with the human immune sera or immune CSF typically showed close to complete overlap in staining obtained with both the monoclonal antibody 4G8 against Aβ and with Thioflavin S. The overlap in staining with Thioflavin S—a fluorescent dye that reacts with β-pleated protein structures—indicated that these human antibodies recognized bona fide brain β-amyloid plaques. We scored the ability of the immune sera to recognize β-amyloid plaques in brain tissue by using the novel TAPIR assay, as disclosed in the instant invention. The 20 patients who generated antibodies against β-amyloid plaques included 6 female and 14 male AD patients with a mean age of 74.6±7.0 (SD) years, baseline Mini Mental State Examination (MMSE) scores of 21.6±3.1 (mean±SD) and a mean duration of disease of 3.6±2.4 (SD) years. Of these 20 patients, 19 observed cases completed the study (6 female, 13 male, mean age 73.4±7.18 years, MMSE 21.3±3.1 points, duration of disease 3.6±2.5 years). The 10 patients without antibodies against β-amyloid included 3 female and 7 male patients, aged 68.8±7.2 years with baseline MMSE scores of 19.9±3.0 and a mean duration of disease of 3.8±2.3 years. Of these 10 patients, 9 observed cases completed the study (3 female, 6 male 68.4±7.1 years, MMSE 19.2±2.5 points, duration of disease 3.4±2.2 years).

Slowed decline of cognitive functions and capacities of daily living: AD patients who generated antibodies against β-amyloid (n=19) performed markedly better on the MMSE one year after the immunization as compared to patients without generation of such antibodies (n=9; p=0.008; ANOVA) (FIG. 2a). As compared to baseline, the patients who generated antibodies against β-amyloid remained unchanged after one year (−1.4±3.5; mean±S.D.; n.s., median=−1.0). In contrast, patients without generation of such antibodies worsened significantly by −6.3±4.0 points (mean±S.D., median=−5.0) on the MMSE scale (p<0.01, Wilcoxon). This magnitude of progression of dementia with deterioration of memory, praxis and orientation, is clinically relevant. The mean value is higher than published rates of decline (−3.9±3.7 MMSE points per year) for the natural history of a large population (n=373) patients with AD (Morris et al., 1993) but both our mean and median values are well within one standard deviation of these published rates of decline This difference is statistically insignificant, and it is most likely caused by the small sample size (n=9) in our group of patients who failed to generate antibodies against β-amyloid. In contrast, the stabilization observed of the group of patients who generated antibodies against β-amyloid differed from published studies of the natural history of AD (Morris et al. 1993). To determine whether the beneficial effects were also noted by the patients' caregivers, we applied the Disability Assessment for Dementia (DAD) rating scale by interviewing caregivers in a double-blinded manner (Gauthier et al., 1997). The DAD specifically assesses such activities as initiation, planning, organization, and performance in basic self care including eating, bathing, grooming, dressing, toileting, as well as instrumental activities of daily living including telephone communication, paying bills, cooking and shopping. Performance in these daily activities was significantly better (p=0.029, ANOVA) in patients who generated antibodies against β-amyloid as compared to patients who did not (FIG. 2b). During one year, the patients who generated antibodies against β-amyloid declined by −2.8±3.8 of 40 points on the DAD as compared to −8.7±10.0 points of the patients who did not. Thus, the cognitive stabilization translated into relevance for daily life.

Relation of clinical outcome to the increase in TAPIR score: To determine whether the clinical outcome of immunotherapy was related to the TAPIR score, we grouped the patients according to the increases in the immunoreactivity scores of β-amyloid plaques on tissue sections. We obtained three groups according to the degree of changes in the immunoreactivity scores: No increase (n=9), intermediate increase (n=13) and strong increase (n=6). It was thus possible to calculate the relationship of the immune response to the clinical outcome (FIG. 3a). Whereas patients with no increases in TAPIR scores worsened markedly, dementia severity in patients with intermediate increases declined only marginally, and patients with strong increases remained stable (p=0.008, ANOVA). These data show a dose-response relationship between serum antibodies against β-amyloid plaques and the clinical outcome. Patients with strong increases in TAPIR scores were essentially protected from disease progression (p=0.003, U-test).

Prevention of disease progression: By using the MMSE to estimate dementia severity, only patients with mild to moderate dementia and with a range of MMSE scores of 16 to 26 points were included in this study. After one year, 6 of 9 (67%) of the patients who failed to generated antibodies against β-amyloid had progressed from mild to moderate dementia to severe dementia with MMSE scores below 14 points. In contrast, only 3 of 19 (16%) of the patients who generated antibodies against β-amyloid progressed from mild to moderate to the severe stage (p<0.01, χ²=7.25; d.f.=1) (FIG. 3b). Moreover, the generation of antibodies against β-amyloid was associated with improved MMSE scores in 4 of 19 (21%) of the patients, whereas no improvements were found when no antibodies against β-amyloid were formed. Halted progression of dementia—as defined by unchanged (±3 points) or higher MMSE scores—was apparent in 12 of 19 (63%) of the patients who generated antibodies against β-amyloid, and in 2 of 9 (22%) of the patients without generation of such antibodies (p<0.05, χ²=4.09; d.f.=1) (FIG. 3c). Notably, two patients who generated antibodies against β-amyloid improved to normal MMSE scores of 28 points (back from 25 points at baseline) to 30 points (back from 24 points at baseline) after one year.

Preserved hippocampal function: Thirteen of 20 (65%) of the patients who generated antibodies against β-amyloid, and 5 of 10 (50%) of the patients who did not, were able to complete the visual paired associated delayed recall test from the Wechsler Memory Scale. This task is a demanding test of hippocampal memory function (Wechsler, 1987). The reasons for not completing (n=12) this test were threefold: The patients were unable to follow the instructions, they were unable to learn the items required for later recall, they simply refused to do this test, or combinations of the above. Upon generation of antibodies against β-amyloid, performance of the subset of patients who completed this test was significantly better (p=0.029, ANOVA) as compared to patients who failed to generated such antibodies (FIG. 4).

Other neuropsychological tests: The generation of antibodies against β-amyloid was generally associated with trends towards better test scores in 6 of 10 assessments including the ADAS-Cog (upon generation of antibodies: −5.5±6.6 points, n=17, mean±S.D, no generation of antibodies: −7.8±4.7, n=6; WMS verbal paired associated delayed recall: −0.3±1.7 points, n=17, vs. 0.6±1.7, n=5; naming: 0.1±0.6 points, n=19, vs. 0.7±0.9, n=9; categorial fluency: −2.0±3.1 points, n=18, vs. 0.5±3.5, n=8; verbal fluency: −3.9±6.8 points, n=18, vs. −7.5±4.7, n=8; CGIC: −0.1±0.7 points, n=18, vs. −1.3±1.1, n=8; CDRS: 0.3±0.5 points, n=18, vs. 0.3±0.4, n=8). It is possible that the kinetics of antibody-related effects vary among distinct brain regions involved in the multiple aspects of cognitive functions assessed by these tests. Such differences would be predicted by the regional differences in brain β-amyloid load following Aβ immunization observed in a recent single case (Nicoll et al. 2003). Larger cohorts of patients with higher statistical power, however, are needed to establish antibody-related statistical differences in a broad range of neuropsychological assessments. Moreover, future continuous follow-up assessments of the current cohorts of patients will be important to determine long-term outcome.

Sustained increases in serum antibodies against β-amyloid: The group of patients who generated antibodies against β-amyloid showed a marked and long-lasting increase in serum antibodies against aggregated Aβ₄₂ in both IgG (FIG. 5a) and IgM (FIG. 5b) classes as measured by ELISA (p=0.005 and p=0.000; ANOVA, two factors, repeated measurements). Titers of both anti-Aβ₄₂-IgG and anti-Aβ₄₂-IgM increased one month after the prime injection, attained a maximum one month after the booster injection, and remained high until month 12. Together, these results show the resustained generation of both IgG and IgM antibodies against aggregated Aβ₄₂ for at least one year. These sustained increases may possibly be related to the fact that the vaccine consisted of highly insoluble aggregates with maintained immunogenicity over long time intervals.

TAPIR assay predicts clinical outcome: If binding to, and removal of, brain β-amyloid is a therapeutic principle in AD, selective antibodies against β-amyloid should have stronger protective effects than anti-Aβ antibodies without the ability to bind β-amyloid. Indeed, we observed that 2 patients with high antibody titers in conventional ELISA assays of anti-Aβ antibodies but with low TAPIR scores against β-amyloid in brain tissue did not experience beneficial clinical effects. In contrast, 3 patients with high TAPIR scores were protected against disease progression—regardless of their low or absent titers in the ELISA assays (p=0.025, χ²=5.0; d.f.=1). Moreover there were no stable or improved patients with high ELISA titers and low TAPIR scores, and there were no worsened patients with high TAPIR scores and low ELISA. The results as reported in the present invention underscore the importance of using appropriate assays for the analysis of clinical outcome, and they clearly demonstrate the necessity for carefully selecting the therapeutically relevant epitope within β-amyloid and its constituents.

Antibodies against β-amyloid can reach the brain: We had available 20 paired CSF samples obtained both at baseline and after the one-year study interval. We found that immune CSF of 4 patients contained antibodies against β-amyloid (FIG. 1b), demonstrating the principle ability for the antibodies to reach the CSF compartment. CSF/serum ratios for albumin were normal in the patients with CSF antibodies against β-amyloid; presence of oligoclonal bands in CSF was observed in one patient. Together, these findings favour passage of antibodies across the blood brain barrier, irrespective of its integrity, over intrathecal production, as an explanation for antibody presence in CSF. The absence of either increased CSF cell counts or increased CSF IgG indices imply that generation of antibodies against β-amyloid is not associated with chronic brain inflammation, although our one-year CSF data can not rule out transient inflammatory episodes during earlier time points within the study period.

Unchanged plasma and CSF levels of Aβ: In our patients, the generation of antibodies against β-amyloid was not associated with major changes in either CSF levels of Aβ₄₀ and Aβ₄₂ (FIG. 6A, B) or in plasma levels of Aβ₄₂ (FIG. 6C). We do not have data on plasma Aβ₄₀ to date. These data argue against the possibility that sequestration of serum Aβ is an underlying principle of the therapeutic effects associated with the generation of antibodies against β-amyloid observed here.

### FIGURES

FIG. 1: Confocal immunofluorescence image of β-amyloid plaques stained by human antibodies against β-amyloid obtained from a patient with AD who participated in this study. (A) Human immune serum: red, (B) human immune CSF: red, (C) monoclonal antibody 4G8: blue, (D) double-staining with human immune CSF and 4G8: purple, (E) thioflavin S: green, (F) double-staining with human immune CSF and thioflavin S: yellow. Scale bar: 20 μm.

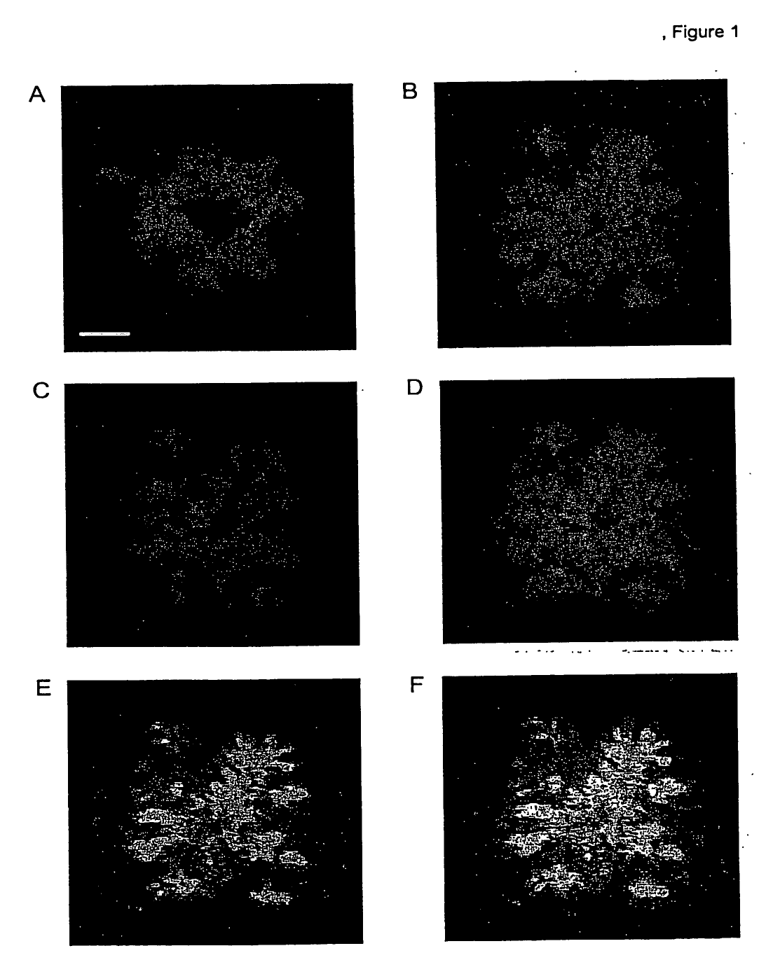

FIG. 2: The presence of antibodies against β-amyloid was associated with slowed decline of both cognitive functions and activities of daily living. (A) Mini Mental State (MMSE) scores. AD patients who responded to β-amyloid immunization with the generation of antibodies against β-amyloid (n=19; filled symbols, solid line) performed significantly better one year after the immunization as compared to the non-responders (n=9; open symbols, dashed line) (*p=0.008; ANOVA). (B) Disability Assessment for Dementia (DAD) rating scale. Patients with antibodies against β-amyloid (n=19; filled symbols, solid line) performed better in daily life, as indicated by the DAD scale, as compared to patients without immune responses (n=9; open symbols, dashed line) (*p=0.030, ANOVA).

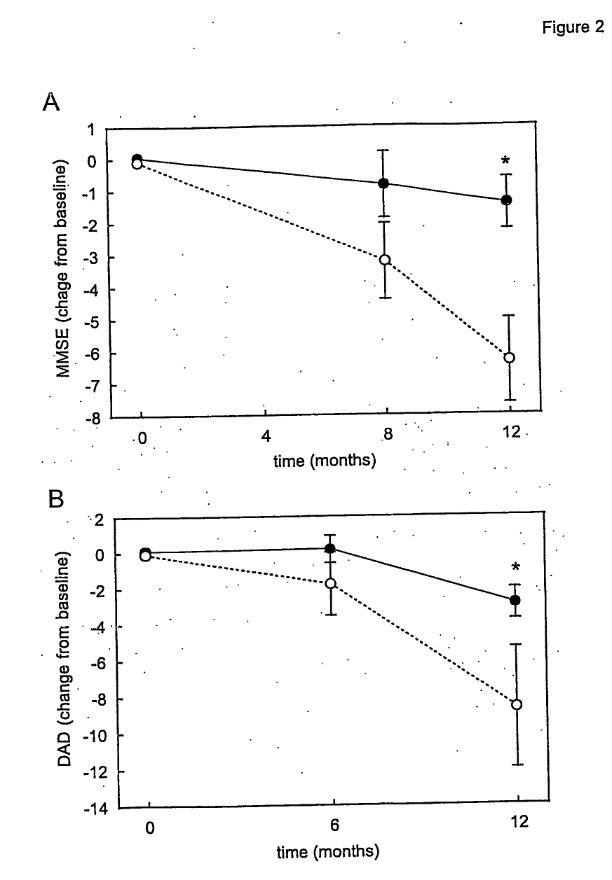

FIG. 3: The degree of the immune response was related to the clinical outcome. (A) Patients were divided in three groups-according to the degree of the immune response, as defined by increases in the β-amyloid immunoreactivity score: no change in immunoreactivity scores (n=9), intermediate responses (n=13) and strong responses (n=6). Whereas patients without immune responses worsened markedly, dementia severity in patients with intermediate increases in β-amyloid immunoreactivity scores declined only marginally, and patients with strong increases remained stable (p=0.008, ANOVA; *p=0.021; **p=0.003; U-tests versus non-responders, respectively). (B) Prevention of disease progression. All study patients who entered this trial had mild to moderate dementia (MMSE 16-26) at baseline (month 0). The presence of antibodies against β-amyloid (filled symbols, solid line) was associated with significantly higher numbers of patients who did not progress to the severe dementia stage as defined by MMSE scores below 14. In contrast, in the absence of an immune response (open symbols, dashed line) the vast majority of patients had progressed to severe dementia within one year (*p<0.01; χ²=7.25; d.f.=1). (C) Cognitive stabilization. MMSE scores were unchanged (±3 points) or higher in 12 of 19 (63%) of the patients with immune responses (solid bar) in contrast to 2 of 9 (22%) of the patients without immune response (open bar) (*p<0.05; χ²=4.09; d.f.=1).

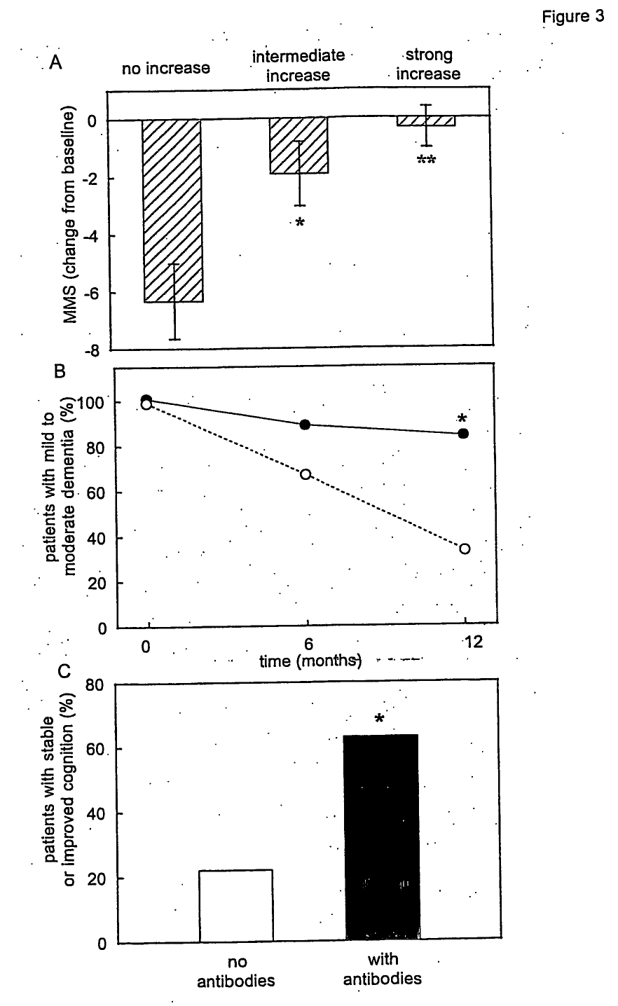

FIG. 4: Preserved hippocampal function. Hippocampal function was tested by the visual paired associated delayed recall test from the Wechsler memory scale. Only two thirds of the study patients in either group were able to complete this task, while the remaining patients were too impaired to complete this test. Performance of the patients with immune responses (n=13; filled symbols, solid line) was significantly better as compared to the patients without immune responses (n=5; open symbols, dashed line) (*p=0.029, ANOVA).

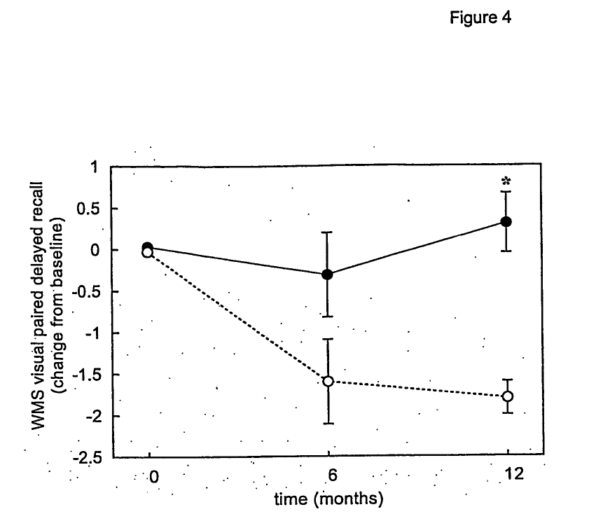

FIG. 5: Sustained increases in serum antibodies. Increases in β-amyloid immunoreactivity scores were associated with (n=20; filled symbols, solid line) marked and long-lasting increases in serum antibodies against Aβ₄₂ in both IgG (A) and IgM (B) classes as measured by ELISA (*p=0.005 and p=0.000; ANOVA), whereas no changes in anti-Aβ₄₂-IgG and anti-Aβ₄₂-IgM titers were observed in the non-responder group (n=10; open symbols, dashed line).

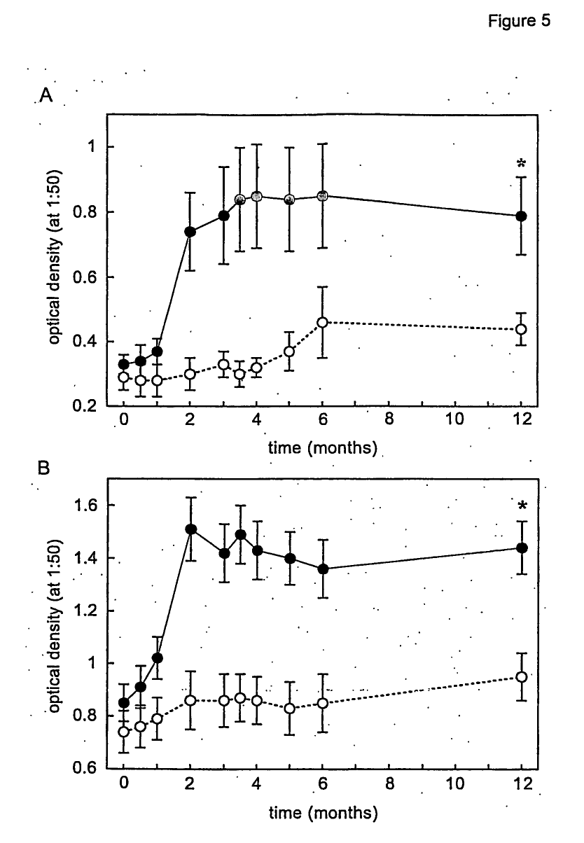

FIG. 6: No differences in CSF or plasma levels of Aβ peptides in patients who generated antibodies against β-amyloid (filled circles) as compared to patients who did not (open circles). A. CSF levels of Aβ₄₂. B. CSF levels of Aβ₄₀. C. Plasma levels of Aβ₄₂. Data are means±S.E.M., n=20 patients who generated antibodies against β-amyloid and n=10 patients who did not.

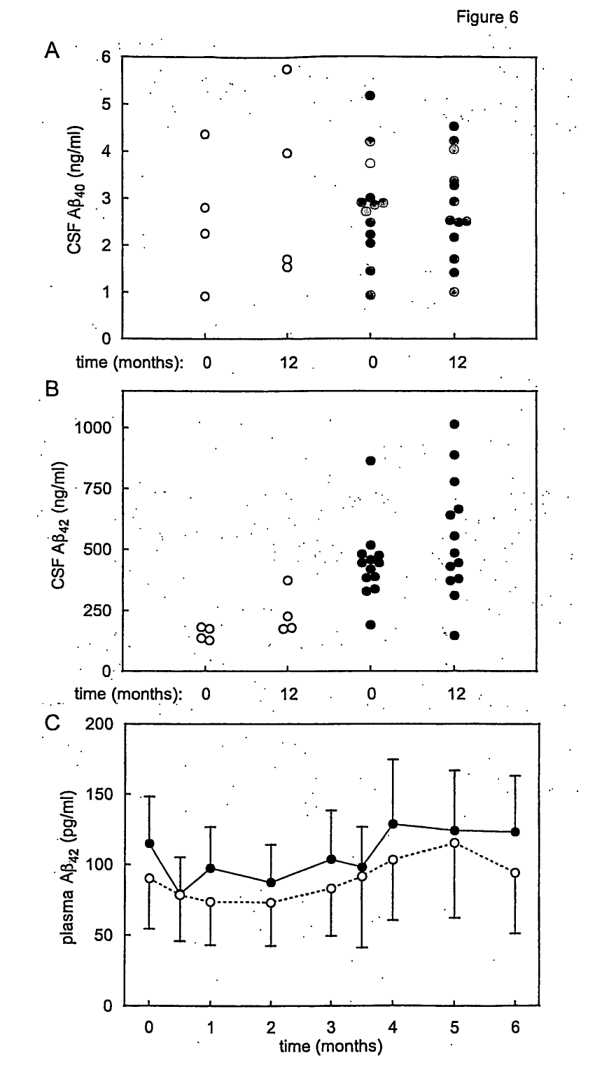

### REFERENCES

Bard, F., et al., “Peripherally administered antibodies against amyloid β-peptide enter the central nervous system and reduce pathology in a mouse model of Alzheimer disease”, Nat. Med. 6:916-919 (2000).

Bard, F., et al., “Epitope and isotype specificities of antibodies to β-amyloid peptide for protection against Alzheimer's disease-like neuropathology”, Proc. Natl. Acad. Sci. USA. 100:2023-2028 (2003).

Chen, G. Q., et al., “A learning deficit related to age and β-amyloid plaques in a mouse model of Alzheimer's disease”, Nature 408:975-979 (2000).

Citron, M., “Alzheimer's disease: treatments in discovery and development”, Nat. Neurosci. 5:1055-1057 (2002).

Consensus recommendations for the post-mortem diagnosis of Alzheimer's disease. The National Institute on Aging, and Reagan Institute Working Group on Diagnostic Criteria for the Neuropathological Assessment of Alzheimer's Disease, Neurobiol. Aging 18:S1-2 (1997).

DeMattos R. B., et al., “Peripheral anti-AP antibody alters CNS and plasma Aβ clearance and decreases brain Aβ burden in a mouse model of Alzheimer's disease”, Proc. Nat. Acad. Sci. U.S.A. 98:8850-8855 (2001).

DeMattos, R. B., et al., “Brain to plasma amyloid-β efflux: a measure of brain amyloid burden in a mouse model of Alzheimer's disease”, Science 295:2264-2267 (2002).

Dodart, J. C., et al., “Immunization reverses memory deficits without reducing brain Aβ burden in Alzheimer's disease model”, Nat. Neurosci. 5:452-457 (2002).

Dominguez, D. I., and De Strooper, B., “Novel strategies provide the real test for the amyloid hypothesis of Alzheimer's disease”, Trends Pharmacol. Sci. 23:324-330 (2002).

Folstein, M. F., et al., “Mini-mental state—A practical method for grading the cognitive state of patients for the clinician”, J. Psychiatr. Res. 12:189-198 (1975).

Gandy, S., “Molecular basis for anti-amyloid therapy in the prevention and treatment of Alzheimer's disease”, Neurobiol. Aging 23:1009-1016 (2002).

Gauthier, S., et al., “Functional disability in Alzheimer's disease”, Int. Psychogeriatr. 9:163-165 (1997).

Geula, C., et al., “Aging renders the brain vulnerable to amyloid beta-protein neurotoxicity”, Nat. Med. 4:827-831 (1998).

Giacobini, E., “Cholinesterase inhibitor therapy stabilizes symptoms of Alzheimer disease”, Alzheimer Dis. Assoc. Disord. 14:S3-10 (2000).

Golde, T. E., “Alzheimer disease therapy: can the amyloid cascade be halted?”, J. Clin. Invest. 111:11-8 (2003).

Gotz, J., et al., “Formation of neurofibrillary tangles in P301L tau transgenic mice induced by Aβ42 fibrils”, Science 293:1491-1495 (2001).

Hardy, J. and Selkoe, D. J., “The amyloid hypothesis of Alzheimer's disease: Progress and problems on the road to therapeutics”, Science 297:353-356 (2002).

Hock, C., et al., “Generation of antibodies specific for β-amyloid by vaccination of patients with Alzheimer disease”, Nat. Med. 8:1270-1275 (2002).

Holcomb, L., et al., “Accelerated Alzheimer-type phenotype in transgenic mice carrying both mutant amyloid precursor protein and presenilin transgenes”, Nat. Med. 4:97-100 (1998).

Ingelsson, M. and Hyman, B. T., “Disordered proteins in dementia”, Ann. Med. 34:259-271 (2002).

Janus, C., et al., “Aβ peptide immunization reduces behavioural impairment and plaques in a model of Alzheimer's disease”, Nature 408:979-982 (2000).

Knopman, D. S., et al., “The Clinician Interview-Based Impression (CIBI): a clinician's global change rating scale in Alzheimer's disease”, Neurology 44:2315-2321 (1994).

Lewis, J., et al., “Enhanced neurofibrillary degeneration in transgenic mice expressing mutant tau and APP”, Science 293:1487-1491 (2001).

McKhann, G., et al., “Clinical diagnosis of Alzheimer's disease: report of the NINCDS-ADRDA Work Group under the auspices of Department of Health and Human Services Task Force on Alzheimer's Disease”, Neurology 34:939-944 (1984).

Morgan, D., et al., “Aβ peptide vaccination prevents memory loss in an animal model of Alzheimer's disease”, Nature 408:982-985 (2000).

Morris, J. C., “The Clinical Dementia Rating (CDR): current version and scoring rules”, Neurology 43:2412-2414 (1993).

Morris, J. C., et al., “The Consortium to Establish a Registry for Alzheimer's Disease (CERAD). Part IV: Rates of cognitive change in the longitudinal assessment of propable Alzheimer's disease”, Neurology 43:2457-2465 (1993).

Morris, J. C., et al., “The Consortium to Establish a Registry for Alzheimer's Disease (CERAD). Part I. Clinical and neuropsychological assessment of Alzheimer's disease”, Neurology 39:1159-1165 (1989).

Naslund, J., et al., “Correlation between elevated levels of amyloid beta-peptide in the brain and cognitive decline”, JAMA 283:1571-1577 (2000).

Nicoll, J. A., et al;., “Neuropathology of human Alzheimer disease after immunization with amyloid-beta peptide: a case report”, Nat. Med. 9:448-452 (2003).

Orgogozo, J.-M., et al., “Subacute meningoencephalitis in a subset of patients with AD after Aβ42 immunization”, Neurology, in press (2003)

Rosen, W. G., et al., “A new rating scale for Alzheimer's disease”, Am. J. Psychiatry 141:1356-1364 (1984).

Schenk, D., et al., “Immunization with amyloid-beta attenuates Alzheimer-disease-like pathology in the POAPP mouse”, Nature 400:173-177 (1999).

Schenk, D., “Amyloid-β immunotherapy for Alzheimer's disease: the end of the beginning”, Nat. Rev. Neurosci. 3:824-828 (2002).

Selkoe, D. J., “Alzheimer's disease: Genes, proteins, and therapy”, Physiol. Rev. 81:741-766 (2001).

Selkoe, D. J., “Deciphering the genesis and fate of amyloid beta-protein yields novel therapies for Alzheimer disease”, J. Clin. Invest. 110:1375-1381 (2002).

Sigurdsson, E. M., et al., “A safer vaccine for Alzheimer's disease?”, Neurobiol. Aging 23:1001-1008 (2002).

Sisodia, S. S. and St. George-Hyslop, P. H., “y-secretase, notch, Aβ and Alzheimer's disease: Where do the presenilins fit in?”, Nat. Rev. Neurosci. 3:281-290 (2002).

Solomon, B., et al., “Disaggregation of Alzheimer beta-amyloid by site-directed mAb”, Proc. Natl. Acad. Sci. U.S.A. 94:4109-4112 (1997).

Steiner, H., and Haass, C., “Intramembraneous proteolysis by presenilins”, Nat. Rev. Mol. Cell Biol. 1:217-224 (2000).

Walter, J., et al., “The cell biology of Alzheimer's disease: uncovering the secrets of secretases”, Curr. Opin. Neurobiol. 11:585-590 (2001).

Wechsler, D., “Wechsler Memory Scale-Revised manual”, San Antonio, Tex.: The Psychological Corporation (1987).

Weiner, H. L. and Selkoe, D. J., “Inflammation and therapeutic vaccination in CNS diseases”, Nature 420:879-884 (2002).

1. A method of monitoring an immunotherapy in a subject suffering from an amyloidogenic disease, comprising the steps of:
(a) obtaining a test sample from a subject being immunized against an amyloid component,
(b) contacting said test sample with an amyloid plaque-containing sample,
(c) determining the level of immunoreactivity of said test sample against amyloid plaques in said amyloid plaque-containing sample, and
(d) comparing said level of immunoreactivity to a reference value representing a known disease or health status, or representing the status prior to onset of said immunotherapy in said subject,
wherein an increase in the level of immunoreactivity of said test sample from said subject undergoing immunotherapy is indicative of a positive clinical outcome of said immunotherapy.

2. The method according to claim 1 wherein said amyloidogenic disease is Alzheimer's disease.

3. The method according to claim 1 wherein said amyloid component is β-amyloid.

4. The method according to claim 1 wherein said test sample is a body fluid.

5. The method according to claim 1 wherein said amyloid plaque-containing sample is obtained from a transgenic non-human animal.

6. The method according to claim 1 wherein said amyloid plaque-containing sample is a tissue section from a transgenic non-human animal.

7. The method according to claim 1 wherein said amyloid plaque-containing sample is a brain tissue section from a non-human animal transgenic for human amyloid precursor protein (APP), or a fragment, or a derivative, or a mutant thereof, and wherein the expression of said transgene results in said non-human animal exhibiting a predisposition to developing amyloid plaques.

8. A method of monitoring an immunotherapy in a subject suffering from a neurodegenerative disease associated with the deposition of abnormal protein aggregates, comprising the steps of:
(a) obtaining a test sample from a subject being immunized against a component of said abnormal protein aggregate,
(b) contacting said test sample with an abnormal protein aggregate-containing sample,
(c) determining the level of immunoreactivity of said test sample against abnormal protein aggregates in said abnormal protein aggregate-containing sample, and
(d) comparing said level of immunoreactivity to a reference value representing a known disease or health status, or representing the status prior to onset of said immunotherapy in said subject,
wherein an increase in the level of immunoreactivity of said test sample from said subject undergoing immunotherapy is indicative of a positive clinical outcome of said immunotherapy.

9. The method according to claim 8 wherein said abnormal protein aggregate-containing sample is obtained from a transgenic non-human animal.

10. The method according to claim 8 wherein said abnormal protein aggregate-containing sample is a tissue section from a non-human animal transgenic for a human protein, or a fragment, or derivative, or a mutant thereof, wherein said human protein is a component of said abnormal protein aggregate, and wherein the expression of said transgene results in said non-human animal exhibiting a predisposition to developing abnormal protein aggregates.

11. A kit for monitoring an immunotherapy in a subject suffering from a neurodegenerative disease associated with the deposition of abnormal protein aggregates, said kit comprising a solid phase containing on its surface an abnormal protein aggregate-containing sample.

12. The kit according to claim 11 wherein said abnormal protein aggregate-containing sample is obtained from a transgenic non-human animal.

13. The kit according to claim 11 wherein said abnormal protein aggregate-containing sample is a tissue section from a transgenic non-human animal.

14. The kit according to claim 11 wherein said abnormal protein aggregate-containing sample is a tissue section from a non-human animal transgenic for a human protein, or a fragment, or derivative, or mutant thereof, wherein said human protein is a component of said abnormal protein aggregate, and wherein the expression of said transgene results in said non-human animal exhibiting a predisposition to developing abnormal protein aggregates.

15. The kit according to claim 14 wherein said human protein is the amyloid precursor protein (APP), or a fragment, or derivative, or mutant thereof.

16. The kit according to claim 11 wherein said neurodegenerative disease is an amyloidogenic disease.

17. The kit according to claim 16 wherein said amyloidogenic disease is Alzheimer's disease.

18. The method according to claim 4 wherein said test sample is serum or cerebrospinal fluid.
